(19) United States
(12) Patent Application Publication
(10) Pub. No.: US 2024/0057983 A1
(43) Pub. Date: Feb. 22, 2024

(54) METERED LIQUID SAMPLE COLLECTION DEVICE

(71) Applicant: Cal Poly Corporation, San Luis Obispo, CA (US)

(72) Inventors: Andres W. Martinez, San Luis Obispo, CA (US); Nathaniel Martinez, San Luis Obispo, CA (US)

(21) Appl. No.: 18/259,983

(22) PCT Filed: Dec. 7, 2021
(86) PCT No.: PCT/US2021/062151

§ 371 Date: Jun. 29, 2023

Related U.S. Application Data
(60) Provisional application No. 63/199,451, filed on Dec. 29, 2020.

(51) Int. Cl. A61B 10/00

(52) U.S. Cl. CPC A61B 10/0051; A61B 10/0064; A61B 10/007; A61B 2010/0006

(57) ABSTRACT

A metered liquid sample collection device includes an absorbent pad, a color indicator concealed from view when the absorbent pad is dry, a mechanism for revealing the color indicator when the appropriate volume of a sample has been collected, and a handle for manipulating the metered liquid sample collection device. The metered liquid sample collection device employs a hydrophilic channel separating the absorbent pad from the color indicator and indicator zone. The mechanism can be a concealed insoluble colored spot deposited on a back face of the indicator zone that only becomes visible when the indicator zone becomes wet, a hydrochromic dye that changes color upon interacting with water, a soluble dye that dissolves into the sample and moves with the sample as it wicks up a zone adjacent the indicator, or an electrical circuit that is completed when the indicator zone becomes wet.

### CROSS REFERENCE TO RELATED APPLICATIONS

This application claims priority under 35 U.S.C. § 119 to provisional patent application U.S. Ser. No. 63/199,451, filed Dec. 29, 2020. The provisional patent application is herein incorporated by reference in its entirety, including without limitation, the specification, claims, and abstract, as well as any figures, tables, appendices, or drawings thereof.

### FIELD OF THE INVENTION

The present invention relates generally to fluid retaining apparatus and/or corresponding method of use in at least the medical industry. More particularly, but not exclusively, the present invention relates to a metered liquid sample collection device.

### BACKGROUND OF THE INVENTION

The background description provided herein gives context for the present disclosure. Work of the presently named inventors, as well as aspects of the description that may not otherwise qualify as prior art at the time of filing, are neither expressly nor impliedly admitted as prior art.

Accurate, rapid and sensitive detection of SARS-CoV-2 is a crucial step in controlling the current pandemic. RT-PCR assays are the gold standard for detection of the virus. The original RT-PCR test relied on a nasal swab for sample collection, but, more recently, it has been demonstrated that saliva is a more convenient sample and does not negatively impact the performance of the test. Collecting saliva, while easier and less invasive than a nasal swab, still has several challenges. For example, subjects may not be able to produce enough saliva for the test, the saliva may be too viscous to be manipulated or measured accurately, or the saliva may contain mucous or other contaminants that could make it unsuitable for testing. These challenges are compounded when the assays are to be performed by automated robots that cannot accurately pipet viscous samples.

Thus, there exists a need in the art for a sample collection device which overcomes these problems.

### SUMMARY OF THE INVENTION

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present invention to collect metered, small volumes (1-100 microliters) of liquid samples. More particularly, a metered liquid sample collection device can be used to collect bodily fluids from humans.

It is a further object, feature, and/or advantage of the present invention to quickly identify, to eliminate, mitigate the spread of, and/or facilitate treatment of infections, viruses, deficiencies, and diseases.

It is still yet a further object, feature, and/or advantage of the present invention to distinguish the invention from use of those other known devices which simply collect saliva in a vial. In such embodiments, it is nearly impossible to know if there is enough saliva. Moreover, the saliva is often contaminated with food and mucus. Further, the present invention aims to distinguish itself from those embodiments which include only a sponge put into the user's mouth and then it is pressed so as to expel the sponge into a tube and/or those devices which are really just a tube with a funnel directing saliva into the collection tube, as these devices also suffer from the aforementioned mucus problems.

The metered liquid sample collection device disclosed herein can be used in a wide variety of applications. For example, the thickness and porosity of the membrane within the metered liquid sample collection device can be varied so as to best suit the particular application in which the sample is being collected. In some embodiments, a 0.2 mm thick paper will not be enough to properly hold saliva, while in others a 0.4 mm thick paper is needed. Sometimes the thickness of the paper will depend on the volume of fluid anticipated to be collected and/or the type of fluid anticipated to be collected (saliva, blood, sweat, tears, urine, etc.).

It is preferred the apparatus be safe, cost effective, and durable. For example, depending on the specific application, some aspects of the metered liquid sample collection device can be reused, stored for later use, and/or designated as a one-time only use. More particularly, in some embodiments, a tube closes after taking the sample, can be dried, and reconstituted later or used right away.

Methods can be practiced which facilitate use, manufacture, assembly, maintenance, and repair of a metered liquid sample collection device which accomplishes some or all of the previously stated objectives. For example, instructions can be provided with the device so that the end user or other person administrating collection of the sample uses the device properly.

The metered liquid sample collection device can be incorporated into systems or kits which accomplish some or all of the previously stated objectives.

According to some aspects of the present disclosure, a metered liquid sample collection device comprises an absorbent pad; an indicator concealed from view when the absorbent pad is dry; a mechanism for revealing the indicator when the appropriate volume of a sample has been collected; and a handle for manipulating the metered liquid sample collection device.

According to some additional aspects of the present disclosure, the metered liquid sample collection device further comprises a hydrophilic channel separating the absorbent pad from the indicator zone, a hydrophobic spacer to separate the handle from the absorbent pad, and/or a region patterned with wax separating the absorbent pad and the indicator from the handle.

According to some additional aspects of the present disclosure, the mechanism is selected from a group consisting of: (1) an insoluble colored spot deposited on a back face of an indicator zone and is only visible when the indicator zone becomes wet, thereby increasing the translucency of the indicator zone, said indicator zone optionally comprising paper and revealing the indicator located there beneath, said insoluble colored spot optionally penetrating through half of the thickness of the indicator zone; (2) a hydrochromic dye that changes color upon interacting with water; (3) a soluble dye that dissolves into the sample and moves with the sample as it wicks up a zone adjacent the indicator; and (4) an electrical circuit that is completed when the indicator zone becomes wet, wherein in some embodiments, completion of the electrical circuit turns on a light, makes a sound, or otherwise produces a signal.

According to some other aspects of the present disclosure, a tube or other collection vessel not integrally formed with the metered liquid sample collection device can be used to help collect the sample and can be shaped so as to substantially match a portion of the metered liquid sample collection device.

According to yet some other aspects of the present disclosure, the device comprises multiple layers. At least one porous membrane may be disposed between at least two of the layers. Wax may also be disposed on at least one of the layers.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE DRAWINGS

Several embodiments in which the present invention can be practiced are illustrated and described in detail, wherein like reference characters represent like components throughout the several views. The drawings are presented for exemplary purposes and may not be to scale unless otherwise indicated.

The patent or application file contains at least one drawing executed in color. Copies of this patent or patent application publication with color drawing(s) will be provided by the Office upon request and payment of the necessary fee.

As shown in the figures, the blue and red wax on the back of the device penetrate through half of the thickness of the paper, so they are not visible from the front unless the paper in the indicator zone becomes wet. The indicator zone changes color when the absorbent pad is filled with sample.

FIG. 1 captures, photographically, a front elevational view of an exemplary metered liquid sample collection device.

FIG. 2 captures, photographically, a rear elevational view of the device of FIG. 1.

FIG. 3A captures, photographically, the device of FIG. 1 before use.

FIG. 3B captures, photographically, the device of FIG. 1 partially filled with sample (˜18 μL).

FIG. 3C captures, photographically, the device of FIG. 1 completely filled with sample (˜20 μL).

FIG. 4 captures, photographically, the device of FIG. 1 stored within a tube in an open configuration. The bottom of the absorbent pad is shaped to fit into the conical 1.5 mL microcentrifuge tube. The handle folds over the edge over the tube so it remains outside of the tube and does not contaminate the sample.

FIG. 5 captures, photographically, the device of FIG. 1 stored within a tube in a closed configuration. As shown, the device is thin enough so the tube can be closed with the handle remaining out of the tube.

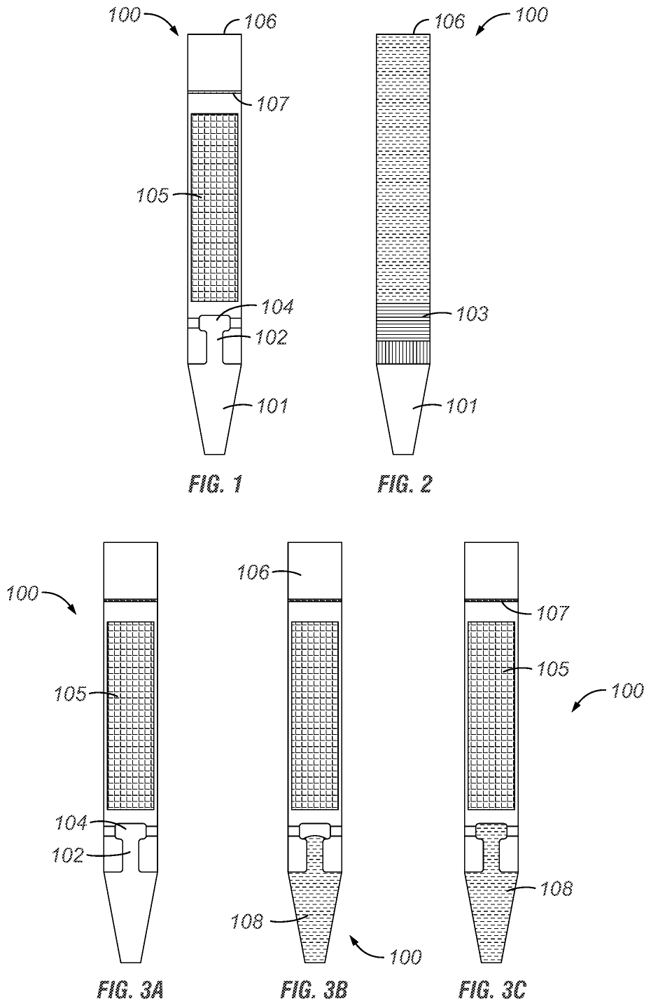

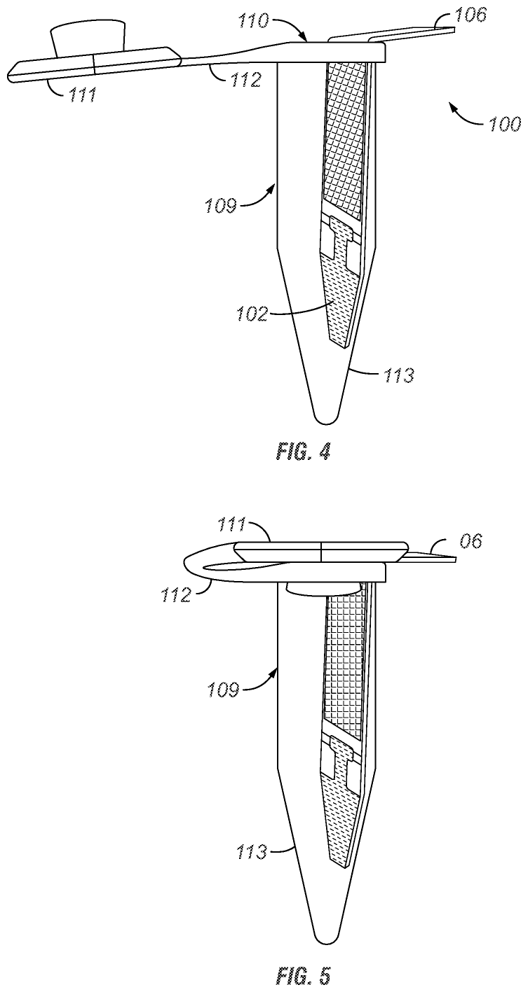

An artisan of ordinary skill need not view, within isolated figure(s), the near infinite number of distinct permutations of features described in the following detailed description to facilitate an understanding of the present invention.

### DETAILED DESCRIPTION OF THE INVENTION

The present disclosure is not to be limited to that described herein. Mechanical, electrical, chemical, procedural, and/or other changes can be made without departing from the spirit and scope of the present invention. No features shown or described are essential to permit basic operation of the present invention unless otherwise indicated.

Referring now to the figures, the metered liquid sample collection device 100 (hereinafter referred to as “device 100”) has an absorbent pad 101 for collecting the sample 108 via capillary wicking, a hydrophilic channel 102 patterned into the device 100 separating the absorbent pad 101 from an indicator zone 104, a mechanism (e.g. 103) for indicating when the appropriate volume of sample 108 has been collected, an intermediate region 105, a handle 106, and a spacer 107.

In some embodiments, the absorbent pad 101 can be a membrane-based microfluidic devices (also called microPADs), such as those described in co-owned U.S. Pat. No. 10,279,344, which is herein incorporated by reference in its entirety. MicroPADs combine some of the capabilities of conventional microfluidic devices, such as the ability to perform multiple quantitative assays simultaneously, with the simplicity of later-flow immunoassays and dipstick assays. MicroPADs are low-cost and easier-to-use alternatives to conventional plastic, glass or silicone-based microfluidic devices.

The sample 108 can be collected directly from the sample source, e.g., by inserting the absorbent pad 101 into the mouth to collect saliva, touching the absorbent pad to the eye to collect tears, touching the absorbent pad 101 to a wound to collect blood or exudate, etc., or indirectly by inserting the absorbent pad 101 into a tube 109 or other collection vessel in which the sample 108 has already been collected. The absorbent pad 101 can be any hydrophilic porous membrane such as cellulose-based paper, nitrocellulose membrane, polyvinylidene difluoride (PVDF) membrane, cellulose acetate membrane. The membrane can be chemically treated to bind to or retain certain components of the sample 108 and stabilize specific biomarkers in the sample 108. For example, cellulose could be oxidized to dialdehyde cellulose, which improves its retention of analytes and stabilizes biomarkers, such as by implementing the method(s) of fabrication described in co-owned U.S. Pre-grant Pub. No. 2019/0300831, which is herein incorporated by reference in its entirety. The absorbent pad 101 can be shaped and sized to fit into any type of tube 109 or container, can determine the volume of sample 108 that is collected, and can allow for improved precision when collecting the sample 108 and to fit into a tube 109 for extraction and processing of the sample 108.

An indicator 103 connected to the absorbent pad 101 tells the user when the absorbent pad 101 has absorbed the predetermined volume of sample 108. The volume of sample 108 can be controlled by the size of the absorbent pad 101 (thickness and surface area). Once the device 100 has collected a sample 108, the sample 108 may be dried on the absorbent pad 101 for long term storage, or the device can be inserted into a tube 109 containing another fluid (e.g., water or buffer) for extraction and sample processing. Some examples of said drying, extraction, and sample processing in connection with the use of microPADs are described in co-owned U.S. Pat. No. 10,343,168, which is herein incorporated by reference in its entirety. The buffer can be specifically tailored to extract and/or process specific types of samples or to test for specific parameters (to identify characteristics of the sample and/or make specific diagnoses). More specifically, the buffer used can be optimized for multiplexing with specific pathogens, including those associated with SARS-CoV-2. The indicator 103 is located at the top of the absorbent pad 101 and is in direct contact with the absorbent pad 101 so that fluid can wick from the absorbent pad 101 into the indicator zone 104. The indicator 103 could be a hydrochromic dye that changes color upon interacting with water, a soluble dye that dissolves into the sample 108 and moves with the sample 108 as it wicks up the indicator zone 104, an insoluble colored spot deposited on a back face of the indicator zone 104 such that it only penetrates through half of the thickness of the indicator zone 104 and is only visible when the indicator zone 104 becomes wet, thus increasing the translucency of the indicator zone 104; or an electrical circuit that is completed when the indicator zone 104 becomes wet and triggers an electrical signal that could turn on a light, make a sound or produce some other kind of signal. For example, when the indicator zone 104 on the front of the device turns pink, this can mean the absorbent pad 101 is saturated with the correct volume of sample 108.

The intermediate region 105 can be patterned with wax so as to separate the absorbent pad 101 and indicator 103 from the handle 106. Instructions or other information may be printed on the intermediate region 105.

The handle 106 for the device is small enough so that when the device 100 is placed in a tube 109, the tube 109 can be closed and the handle 106 remains outside of the tube 109. Such a configuration helps prevent contamination of the sample 108 and facilitates removal of the device from the tube 109 after processing.

The handle 106 can be further separated from the absorbent pad 101 by a spacer 107 to prevent contamination of the sample 108 and facilitate sample collection. The handle 106 can be used to manipulate the device 100. The handle 100 will remain outside the extraction tube 109 so that the device 100 can be removed from the tube 109 without contaminating the sample 108 in the tube 109.

One application of the device is in the collection of saliva samples for the detection of SARS-CoV-2. For such an application, most aspects if not all of the aspects of the device 100 (e.g., the handle 106, indicator 103 and absorbent pad 101) can be made out of Whatman 3MM chromatography paper. The handle 106 and indicator 103 are patterned with wax to make these regions impermeable to aqueous solutions. The indicator 103 is made by patterning colored wax on one face of the paper and allowing it to penetrate only partially through the thickness of the paper. Saliva is collected by inserting the device 100 into the mouth to collect saliva. The device is then inserted into a tube 109 containing a fixed volume of water.

In some embodiments, the tube 109 includes an inlet 110, a cap 111, a hinge 112 allowing for movement of the cap 111 from an open position to a closed position, and a conical portion 113. In such embodiments, the tube 109 can be capped with the handle 106 remaining outside the tube 109. The tube 109 can then be heated to inactivate any pathogens in the sample 108 and release viral RNA into the fluid. The tube 109 can then be opened by pushing up on the cap 111 and allowing the cap to rotate about hinge 111 such that the device 100 can be removed therefrom. The device 100 can then be discarded. The solution in the tube 109 is analyzed by RT-PCR. Results are comparable to assays conducted with saliva collected in a tube.

From the foregoing, it can be seen that the present invention accomplishes at least all of the stated objectives.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 1
List of Reference Characters

| | |
|---|---|
| 100 | metered liquid sample collection device |
| 101 | absorbent pad |
| 102 | hydrophilic channel |
| 103 | indicator |
| 104 | indicator zone |
| 105 | intermediate region |
| 106 | handle |
| 107 | spacer |
| 108 | sample |
| 109 | tube |
| 110 | inlet |
| 111 | cap |
| 112 | hinge |
| 113 | conical portion |

### Glossary

Unless defined otherwise, all technical and scientific terms used above have the same meaning as commonly understood by one of ordinary skill in the art to which embodiments of the present invention pertain.

The terms “a,” “an,” and “the” include both singular and plural referents.

The term “or” is synonymous with “and/or” and means any one member or combination of members of a particular list.

The terms “invention” or “present invention” are not intended to refer to any single embodiment of the particular invention but encompass all possible embodiments as described in the specification and the claims.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present invention is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

Additional elements and/or steps may be added to various aspects of the invention and/or some disclosed elements and/or steps may be subtracted from various aspects of the invention without departing from the scope of the invention. Singular elements/steps imply plural elements/steps and vice versa. Some steps may be performed serially, in parallel, in a pipelined manner, or in different orders than disclosed herein. Many other variations are possible which remain within the content, scope, and spirit of the invention, and these variations would become clear to those skilled in the art after perusal of this application.

The present disclosure is further defined by the following numbered paragraphs:
1. A metered liquid sample collection device (100) comprising:
an absorbent pad (101);
a color indicator (103) concealed from view when the absorbent pad (101) is dry; a mechanism for revealing the color indicator (103) when the appropriate volume of a sample (108) has been collected; and
a handle (106) for manipulating the metered liquid sample collection device (100).
2. The metered liquid sample collection device (100) of paragraph 1 further comprising a hydrophilic channel (102) separating the absorbent pad (101) from the color indicator (103) and indicator zone (104).
3. The metered liquid sample collection device (100) of paragraph 2 wherein the mechanism is an insoluble colored spot deposited on a back face of an indicator zone (104) and is only visible when the indicator zone (104) becomes wet, thereby increasing the translucency of the indicator zone (104).
4. The metered liquid sample collection device (100) of paragraph 3 wherein the indicator zone (104) comprises chromatography paper.
5. The metered liquid sample collection device (100) of paragraph 4 wherein a thickness of the chromatography paper depends on a type of the sample to collected, said type selected from the group consisting of: blood, sweat, saliva, urine, and tears.
6. The metered liquid sample collection device (100) of paragraph 5 wherein the insoluble colored spot penetrates through half of the thickness of the indicator zone (104).
7. The metered liquid sample collection device (100) of paragraph 2 wherein the mechanism is a hydrochromic dye that changes color upon interacting with water.
8. The metered liquid sample collection device (100) of paragraph 2 wherein the mechanism is a soluble dye that dissolves into the sample (108) and moves with the sample (108) as it wicks up a zone adjacent the color indicator (103).
9. The metered liquid sample collection device (100) of any one of paragraphs 1-8 wherein the absorbent pad (101) is shaped to resemble a surface of a conical portion (113) of the tube (109) or other collection vessel so as to substantially cover said portion (113) when placed therein.
10. The metered liquid sample collection device (100) of any one of paragraphs 1-9 wherein a spacer (107) to separate the handle (106) from the absorbent pad (101).
11. A method of using the metered liquid sample collection device of any one of paragraphs 1-10 comprising:
collecting the sample (108) with the metered liquid sample collection device (100) or the tube (109);
subsequently placing the metered liquid sample collection device (100) into the tube (109) or other collection vessel; and
(i) drying the sample (108) on the absorbent pad (101) for long term storage or (ii) extracting an processing the sample (108) using a buffer.
12. The method of paragraph 11 wherein the handle (106) remains outside of the tube (109) or other collection vessel when the metered liquid sample collection device (100) is placed and capped therewithin.
13. The method of paragraph 12 wherein the metered liquid sample collection device (100) can be removed from the tube (109) or other collection vessel without contaminating the sample (108) in the tube (109) or other collection vessel.
14. A kit comprising the metered liquid sample collection device (100) of any one of paragraphs 1-10, said kit comprising a tube (109) or other collection vessel not integrally formed with the metered liquid sample collection device.
15. The kit of paragraph 14 further comprising instructions for use of the metered liquid sample collection device printed on the metered liquid sample collection device (100).
16. A metered liquid sample collection device (100) comprising:
an absorbent pad (101) located at a first end of the metered liquid sample collection device (100);
an indicator (103) that operatively indicates when the appropriate volume of a sample (108) has been collected;
a handle (106) for manipulating the metered liquid sample collection device (100) located at a second end of the metered liquid sample collection device (100) opposite the first end; and
an intermediate, hydrophobic region (105) separating the absorbent pad (101) and the indicator (103) from the handle (106).
17. The metered liquid sample collection device (100) of paragraph 16 wherein the intermediate, hydrophobic region (105) is patterned with wax.
18. The metered liquid sample collection device (100) of any one of paragraphs 16-17 wherein the intermediate, hydrophobic region (105) comprises plastic.
19. A metered liquid sample collection device (100) comprising:
an absorbent pad (101);
an indicator (103) inactivated when the absorbent pad (102) is dry;
a mechanism for activating the indicator (103) when the appropriate volume of a sample (108) has been collected; and
a handle (106) for manipulating the metered liquid sample collection device (100).
20. The metered liquid sample collection device (100) of paragraph 19 wherein:
the mechanism comprises an electrical circuit that is completed when an indicator zone (104) associated with the indicator (103) becomes wet; and
   completion of the electrical circuit turns on a light, makes a sound, or otherwise produces a signal.

What is claimed is:

1. A metered liquid sample collection device comprising:
an absorbent pad;
a color indicator concealed from view when the absorbent pad is dry;
a mechanism for revealing the color indicator when the appropriate volume of a sample has been collected; and
a handle for manipulating the metered liquid sample collection device.

2. The metered liquid sample collection device of claim 1 further comprising a hydrophilic channel separating the absorbent pad from the color indicator and indicator zone.

3. The metered liquid sample collection device of claim 2 wherein the mechanism is an insoluble colored spot deposited on a back face of an indicator zone and is only visible when the indicator zone becomes wet, thereby increasing the translucency of the indicator zone.

4. The metered liquid sample collection device of claim 3 wherein the indicator zone comprises chromatography paper.

5. The metered liquid sample collection device of claim 4 wherein a thickness of the chromatography paper depends on a type of the sample to collected, said type selected from the group consisting of: blood, sweat, saliva, urine, and tears.

6. The metered liquid sample collection device of claim 5 wherein the insoluble colored spot penetrates through half of the thickness of the indicator zone.

7. The metered liquid sample collection device of claim 2 wherein the mechanism is a hydrochromic dye that changes color upon interacting with water.

8. The metered liquid sample collection device of claim 2 wherein the mechanism is a soluble dye that dissolves into the sample and moves with the sample as it wicks up a zone adjacent the color indicator.

9. The metered liquid sample collection device of claim 1 wherein the absorbent pad is shaped to resemble a surface of a conical portion of the tube or other collection vessel so as to substantially cover said portion when placed therein.

10. The metered liquid sample collection device of claim 1 wherein a spacer to separate the handle from the absorbent pad.

11. A method of using the metered liquid sample collection device of claim 1 comprising:
collecting the sample with the metered liquid sample collection device or the tube;
subsequently placing the metered liquid sample collection device into the tube or other collection vessel; and
(i) drying the sample on the absorbent pad for long term storage or (ii) extracting an processing the sample using a buffer.

12. The method of claim 11 wherein the handle remains outside of the tube or other collection vessel when the metered liquid sample collection device is placed and capped therewithin.

13. The method of claim 12 wherein the metered liquid sample collection device can be removed from the tube or other collection vessel without contaminating the sample in the tube or other collection vessel.

14. A kit comprising the metered liquid sample collection device of claim 1, said kit comprising a tube or other collection vessel not integrally formed with the metered liquid sample collection device.

15. The kit of claim 14 further comprising instructions for use of the metered liquid sample collection device printed on the metered liquid sample collection device.

16. A metered liquid sample collection device comprising:
an absorbent pad located at a first end of the metered liquid sample collection device;
an indicator that operatively indicates when the appropriate volume of a sample has been collected;
a handle for manipulating the metered liquid sample collection device located at a second end of the metered liquid sample collection device opposite the first end; and
an intermediate, hydrophobic region separating the absorbent pad and the indicator from the handle.

17. The metered liquid sample collection device of claim 16 wherein the intermediate, hydrophobic region is patterned with wax.

18. The metered liquid sample collection device of claim 16 wherein the intermediate, hydrophobic region comprises plastic.

19. A metered liquid sample collection device comprising:
an absorbent pad;
an indicator inactivated when the absorbent pad is dry;
a mechanism for activating the indicator when the appropriate volume of a sample has been collected; and
a handle for manipulating the metered liquid sample collection device.

20. The metered liquid sample collection device of claim 19 wherein:
the mechanism comprises an electrical circuit that is completed when an indicator zone associated with the indicator becomes wet; and
completion of the electrical circuit turns on a light, makes a sound, or otherwise produces a signal.
